(19) United States
(12) Patent Application Publication
(10) Pub. No.: US 2023/0082235 A1
(43) Pub. Date: Mar. 16, 2023

(54) SYSTEM AND METHOD FOR TOPICAL SPRAY, SALVE, AND TINCTURE

(71) Applicant: Christina Nadine Evans, Chicago, IL (US)

(72) Inventor: Christina Nadine Evans, Chicago, IL (US)

(21) Appl. No.: 17/943,853

(22) Filed: Sep. 13, 2022

Related U.S. Application Data
(60) Provisional application No. 63/244,679, filed on Sep. 15, 2021.

(51) Int. Cl. A61K 36/28; A61K 36/738; A61K 36/889; A61K 9/70

(52) U.S. Cl. CPC A61K 36/28; A61K 36/738; A61K 36/889; A61K 9/7015

(57) ABSTRACT

A system and method for a salve, spray, and tincture that help with rheumatism, arthritis, gout, and any other inflammation in joints or muscles whereby decarboxylation helps activate the herbs to help with how fast the relief works, while being infused in coconut oil, whereby the spray has wheatgerm oil for pain and safflower oil and eves primrose oil helps dissolve lactic and uric acid build up in joints that cause arthritis and the tincture whereby the tincture is created from the remaining herbs in the creation of the salve and sat in Everclear.

### CROSS REFERENCE TO RELATED APPLICATIONS

This application claims priority to U.S. Provisional Patent No. 63/244,679 filed on Sep. 15, 2021 which is incorporated in its entirety.

### FIELD OF THE DISCLOSURE

This disclosure relates generally to novel formulations for the purpose of alleviating physical and mental pain and more particularly to a topical spray, ointment, and tincture that relieves immobility in joints affected by arthritis as well as swelling and pain.

### BACKGROUND

Arthritis is the swelling and tenderness of one or more of your joints. The main symptoms of arthritis are joint pain and stiffness, which typically worsen with age. The most common types of arthritis are osteoarthritis and rheumatoid arthritis. Many people currently are suffering from arthritis. Typically, products like Icy Hot and Biofreeze are used to mask the problems but only numb the pain and the pain eventually resumes. Thus exists the need for a product that actually helps with rheumatism, arthritis, gout, and any other inflammation in joints or muscles and bringing mobility back to affected joints using all natural herbs for the salve and all natural oils for the spray with no added chemicals.

### SUMMARY

The present invention is directed to medicinal treatment for a user or patient with spray and salve for external use on the user or patient, and a tincture for internal use thus providing a unique combination that works together to alleviate many health problems such as that rheumatism, arthritis, gout, and any other inflammation in joints or muscles. with the user. The spray is a 15 ml amber colored spray bottle that is filled with 3 parts of oil, safflower oil 5 ml, eves primrose oil 5 ml, and wheatgerm oil 5 ml. The salve is 1 tablespoon of burdock root, red clover, yellow dock, dandelion root, chamomile, cayenne, meadowsweet, bugleweed and nettle put into a 16 oz mason jar to decarboxylate. Coconut oil is melted down and poured into the jar filled with herbs to then infuse the herbs into the coconut oil. A tincture is created from the remaining coconut oil where the coconut oil is combined with high proof alcohol for a predesignated amount of time.

### DETAILED DESCRIPTION

In the Summary above and in this Detailed Description, and the claims below, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises,” and grammatical equivalents thereof are used herein to mean that other components, ingredients, steps, among others, are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The present description is directed to a system and method for a spray and salve that helps with arthritis, relieving pain, and bringing mobility back to affected joints using all natural herbs for the salve and all natural oils for the spray with no added chemicals. The spray and salve may be used in combination where the spray is sprayed onto the affected area then the salve is positioned on the affected area or vice versa or separately.

The spray may be a combination of equal parts safflower oil, primrose oil, and wheatgerm oil such as but not limited to 5 ml safflower oil, 5 ml eves primrose oil, and 5 ml wheatgerm oil. The wheatgerm oil is for pain and the safflower oil and eves primrose oil help dissolve lactic and uric acid build up in joints that cause arthritis. The components of the spray once formed may be inserted into a spray bottle. The spray bottle may include a head, a reservoir, and a nozzle. The head may include an internal chamber and a pump. The nozzle may further be configured to be an outlet for the fluid. The nozzle may be connected to a nozzle attachment and a trigger mechanism. In one or more embodiments when the trigger mechanism is depressed, a pump positioned inside of the head may be activated to force fluid from the reservoir into a spray bottle tube then into an internal chamber of the head, and then out through the nozzle. The pump may be an automatic or mechanical pump, such as a compressor or a manual pump. The spray may be applied to the affected area of the user. In a preferred embodiment, the spray bottle is a 15 ml amber colored spray bottle.

The salve may be a combination of herbs such as burdock root, red clover, yellow dock, dandelion root, chamomile, cayenne, meadowsweet, bugleweed, and nettle. The herbs may be fresh, dry, ground, or chopped. In a preferred embodiment there may be one tablespoon of each of the herbs. The components may then proceed through a decarboxylation process whereby they are placed in a container and heated in an oven or heated in a decarboxylator for a predetermined amount of time or stored in a mason jar for a predetermined amount of time. Coconut oil may then be melted down and poured onto the herbs to infuse the herbs into an amount of coconut oil. This decarboxylation process activates the herbs to help with how fast the relief works. The salve may then be taken out of the mason jar or other container and applied to the affected area of the user. The salve may be strained before applying to the affected area of the user.

The creation of the salve leaves herbs have been infused in with a number of particulates still lingering in the coconut oil. The herbs that are infused with the coconut oil is then transferred to a mason jar to sit in 150 proof Everclear alcohol with a ratio of 1:4 alcohol to herbs. This mixture may then be stored 3 weeks or longer depending on the quality needed. Once the necessary time has passed, the mixture may be strained with a micro strainer and then placed into mason jar. When needed, the created tincture may be placed into a plurality of eye droppers. The tincture is placed in a UV protected dropper bottle

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. A system for medicinal treatment for internal use and external use on a body.

2. The system for medicinal treatment of claim 1 comprising a spray having safflower oil, eves primrose oil, and wheatgerm oil.

3. The system for medicinal treatment of claim 2, wherein the safflower oil, the eves primrose oil, and the wheatgerm oil are of equal parts.

4. The system for medicinal treatment of claim 3, wherein the equal parts is 5 milliliters each.

5. The system for medicinal treatment of claim 1, comprising a salve that is a combination of herbs including burdock root, red clover, yellow dock, dandelion root, chamomile, cayenne, meadowsweet, bugleweed, and nettle.

6. The system for medicinal treatment of claim 5, wherein the burdock root, the red clover, the yellow dock, the dandelion root, the chamomile, the cayenne, the meadowsweet, the bugleweed, and the nettle are of equal parts.

7. The system for medicinal treatment of claim 6, wherein the equal parts are one tablespoon.

8. The system for medicinal treatment of claim 5, wherein the herbs are fresh, dry, ground, or chopped.

9. The system for medicinal treatment of claim 5, wherein the herbs have been utilized in a decarboxylation process.

10. The system for medicinal treatment of claim 9, wherein coconut oil has been melted down and poured onto the herbs to infuse the herbs into an amount of the coconut oil.

11. The system for medicinal treatment of claim 10, further comprising a tincture wherein the tincture is created from remaining herbs from the infused coconut oil.

12. The system for medicinal treatment of claim 11, wherein the remaining herbs is mixed with high proof alcohol.

13. The system for medicinal treatment of claim 12, wherein the high proof alcohol is 150 proof.

14. The system for medicinal treatment of claim 12, wherein the remaining herbs has been mixed with the high proof alcohol for a period of three weeks.

15. A method for medicinal treatment on a body, the method including creating a spray and salve for external use on the body and a tincture for internal use on the body.

16. The method of claim 15 further comprising: creating the spray with equal parts of safflower oil, eves primrose oil, and wheatgerm oil.

17. The method of claim 16, creating the salve with a combination of herbs including burdock root, red clover, yellow dock, dandelion root, chamomile, cayenne, meadowsweet, bugleweed, and nettle;
placing the combination of herbs in a container; and
placing the combination of herbs through a decarboxylation process by heating in an oven or decarboxylator for a period of time.

18. The method of claim 17 further comprising melting coconut oil; and
pouring the combination of herbs to infuse the combination of herbs into the coconut oil.

19. The method of claim 18 further comprising mixing reamining herbs from infusion, with high proof alcohol infusion with a high proof alcohol to create a mixture; and
storing the mixture for a period of at least three weeks.

20. The method of claim 19 further comprising straining the mixture with a micro strainer; and
placing the mixture into one or more 15 ml bottles.
